(19) United States
(12) Patent Application Publication
(10) Pub. No.: US 2013/0345160 A1
(43) Pub. Date: Dec. 26, 2013

(54) GLYCYRRHIZIN HIGH-CONCENTRATION PREPARATION

(71) Applicant: NIPPON ZOKI PHARMACEUTICAL CO., LTD., OSAKA-SHI (JP)

(72) Inventors: Taro YOSHIKAWA, Osaka (JP); Satoshi HANAOKA, Osaka (JP)

(73) Assignee: NIPPON ZOKI PHARMACEUTICAL CO., LTD., OSAKA-SHI (JP)

(21) Appl. No.: 14/010,141

(22) Filed: Aug. 26, 2013

Related U.S. Application Data
(63) Continuation-in-part of application No. 10/566,588, filed on Mar. 9, 2006.

(30) Foreign Application Priority Data
Aug. 12, 2003 (JP) 2003-292135

(51) Int. Cl. A61K 31/704; A61K 31/198; A61K 9/00

(52) U.S. Cl. CPC A61K 31/704; A61K 9/0019; A61K 31/198
USPC 514/33

(57) ABSTRACT

To provide a glycyrrhizin/aminoacetic acid/cysteine combination drug containing an active ingredient in high concentration and excelling in stability and safety. Stability at compounding of active ingredient in high concentration has been improved by avoiding the addition of sulfite salts having been used as a stabilizer in existing products. As glycyrrhizin high-concentration preparations, there is provided a drug composition comprising 8 to 16 mg/mL of glycyrrhizin, 3 to 6 mg/mL of cysteine and 80 to 160 mg/mL of aminoacetic acid wherein no sulfite salts are added as an additive, and pH is 6 to 7.

This is a Continuation-in-Part of application Ser. No. 10/566,588 filed Mar. 9, 2006, which in turn is a National Phase Application of PCT/JP2004/011462, filed on Aug. 10, 2004, which claims the benefit of Japanese Patent Application No. 2003-292135, filed on Aug. 12, 2003. The disclosure of the prior applications is hereby incorporated by reference herein in its entirety.

### TECHNICAL FIELD

The present invention relates to a pharmaceutical composition containing high concentrations of glycyrrhizin, cysteine and aminoacetic acid (glycine) which are useful as drugs for hepatic diseases or for allergy.

### BACKGROUND ART

It has been known already that glycyrrhizins have various kinds of pharmacological actions such as anti-cortisone action, decholesterolizing action, anti-allergic action, anti-inflammatory action, detoxifying actin and reparative action for gastric ulcer and their safety has been also confirmed whereby glycyrrhizin preparations containing the same as an effective ingredient have been widely used as remedies for various diseases. In recent years, efficacy of a megadose of glycyrrhizin by intravenous injection to chronic hepatic diseases has been reported whereby utility of glycyrrhizin preparations has been reconsidered.

In general, it is often in the case of drugs for the treatment of hepatic disease that drug is continuously administered for a relatively long period. A combination drug of glycyrrhizin, aminoacetic acid and cysteine which has now been available in the market (trade name: Stronger Neo-Minophagen C) is an injection preparation in which 2.65 mg/mL of monoammonium glycyrrhizinate (2 mg/mL as glycyrrhizin), 1 mg/mL of cysteine hydrochloride (0.77 mg/mL as cysteine) and 20 mg/mL of aminoacetic acid are compounded. Although its dose is appropriately increased or decreased depending upon age and symptom, it is intravenously injected or intravenously dripped at the dose of 40 to 60 mL a day once daily (which may be increased up to 100 mL) to chronic hepatic diseases. Such an administration in large dose results in a problem that not only pain to a patient is resulted upon administration but also administration of day after day for long time makes the tissue of the injected site thick. There are other problems that glycyrrhizin which is a component compounded in this preparation is precipitated or that cysteine hydrochloride is apt to be degraded and is unstable (refer to Patent Document 1) and, in the existing injection preparations, a sulfite (0.8 mg/mL of sodium sulfite) is added as a stabilizer (refer to Non-Patent Document 1). However, there is a report that a sulfite is an attractant for onset of asthma (refer to Non-Patent Document 2) and it has also been reported as a food additive which induces allergy (refer Non-Patent Documents 3 and 4). Accordingly, pharmaceutical preparations containing them in high concentrations have a problem in terms of safety.
Patent Document 1: Japanese Patent Laid-Open No. 2002/065,808, page 2, column 0004
Non-Patent Document 1: Package Insert for a Drug “Stronger Neo-Minophagen C” (prepared by K. K. Minophagen Seiyaku)
Non-Patent Document 2: Tatsuo Sakamoto: “Food Additives and Asthma (Mainly Concerning Sulfites), Airway Allergy” '96, Medical View Firm; page 151, 1996
Non-Patent Document 3: Yoichi Kawano: Fundamentals and Clinics of Food Allergy (Allergenicity of Food), Allergology & Immunology, 4(6), pages 741 to 745, 1997
Non-Patent Document 4: Masataka Michibata: Self-Control of Steroids, My means, Climics & Drug Therapy, 16(3), pages 226 to 230, 1997

### DISCLOSURE OF THE INVENTION

### Problems that the Invention is to Solve

As mentioned above, in administration of a combination drug of glycyrrhizin, aminoacetic acid and cysteine, there has been a demand for making the burden on patients as little as possible such as pain and tissue thickening of the injected site upon megadose by intravenous injection and the like, whereby the present inventors have carried out intensive studies for high-concentration preparations where a pharmaceutical effect can be expected by administration of a small dose and where stability and safety are high.

An object of the present invention is to provide a combination drug of glycyrrhizin, aminoacetic acid and cysteine where effective ingredients are compounded in higher concentrations than the conventional product and wherein the combination also has high stability and safety. Even when concentrations of the compounded components in the conventional product are merely made high, degradation, precipitation and the like of effective ingredients are resulted and no sufficient stability is available. Further, problem in terms of safety due to the sulfite contained therein also resulted.

### BRIEF SUMMARY OF THE INVENTION

In order to solve the aforementioned problem, the present inventors have carried out intensive studies and found that, when sodium sulfite which has been used as a stabilizer in the conventional product is not used, stability when effective ingredients are compounded in high concentrations are improved. In this way, the present invention has been achieved through compounding a combination drug of glycyrrhizin, aminoacetic acid and cysteine in which effective ingredients are contained in higher concentrations than the conventional product, and which is able to be prepared with high safety.

In the pharmaceutical composition of the present invention, a sulfite which has been used as a stabilizer in the conventional product is not used, and also the pharmaceutical composition is adjusted in specific range of pH and, as a result, precipitate of glycyrrhizin which is compounded in a high concentration is not produced, reduction in the amount of cysteine therein is low and stability is enhanced.

### BRIEF DESCRIPTION OF THE DRAWINGS

FIG. 1 is a result where the pharmaceutical compositions were stored in a chamber of constant temperature and humidity at 40° C. and 75% RH (Relative Humidity) and, after six months, the amount of L-cysteine in each preparation was quantified by high-performance liquid chromatography (HPLC).

### DETAILED DESCRIPTION OF THE INVENTION

The present invention relates, in a glycyrrhizin preparation where glycyrrhizin or a pharmacologically acceptable salt is an effective ingredient, to a combination drug of glycyrrhizin, aminoacetic acid and cysteine in high concentrations which is characterized in that cysteine and aminoacetic acid are contained in addition to the aforementioned ingredient and that pH is 6 to 7, and no sulfite is contained therein.

Glycyrrhizin which is an effective ingredient of the pharmaceutical composition of the present invention is able to be prepared by extracting from licorice root or that which has been available in the market may be used as well. Glycyrrhizin is also called glycyrrhizinic acid and, in the present invention, it includes a pharmaceutically acceptable salt of glycyrrhizin. Examples of the pharmaceutically acceptable salt are salts with acid or base including ammonium salt such as monoammonium glycyrrhizinate and alkali metal salt such as disodium glycyrrhizinate, trisodium glycyrrhizinate and dipotassium glycyrrhizinate.

Further, cysteine and aminoacetic acid (glycine) in the present invention also include pharmaceutically acceptable salts thereof and their examples cover both kinds of salts of an acid addition salt such as that with hydrochloric acid or malic acid and a base addition salt such as that with alkali metal (e.g., sodium), alkali earth metal, ammonium and nitrogen-containing organic base. A preferred salt of cysteine is a hydrochloride. Moreover, a hydrate of cysteine, aminoacetic acid or a salt thereof such as cysteine hydrochloride monohydrate and sodium aminoacetate hydrate may be also included as cysteine and aminoacetic acid of the present invention. There are optically active substances in cysteine and any of L- and racemic substances may be used and, preferably, L-cysteine may be used.

The preferred compounding amounts in the pharmaceutical composition of the present invention are in amounts of from 4- to 8-fold of those of the effective ingredients in the aforementioned conventional preparation. Thus, it is a pharmaceutical composition containing 8 to 16 mg/mL of glycyrrhizin, 4 to 8 mg/mL of cysteine hydrochloride (3 to 6 mg/mL as cysteine) and 80 to 160 mg/mL of aminoacetic acid. Preferably, it is a pharmaceutical composition containing 8 to 12 mg/mL of glycyrrhizin, 4 to 6 mg/mL of cysteine hydrochloride (3 to 5 mg/mL as cysteine) and 80 to 120 mg/mL of aminoacetic acid. Particularly preferably, it is a pharmaceutical composition containing 10 mg/mL of glycyrrhizin, 5 mg/mL of cysteine hydrochloride and 100 mg/mL of aminoacetic acid. Incidentally, each of the aforementioned values for the concentrations (mg/mL) is in accordance with the regulation for standard values mentioned in General Rule 18 of the Japanese Pharmacopoeia (the 14th revision) and is a value rounding off the first decimal place. Furthermore, it is preferable that the glycyrrhizin and the cysteine are monoammonium glycyrrhizinate and cysteine hydrochloride, respectively.

The pharmaceutical composition of the present invention may be produced that predetermined amounts of sodium hydroxide, grycyrrhizine, cysteine and amino acetic acid are successively dissolved in water, then adjusted at predetermined pH. Any acid or alkaline may be used as a pH adjuster, and hydrochloric acid or sodium hydroxide is preferred. Preferably, the pH of pharmaceutical composition may be adjusted at 6 to 7. Furthermore, all of the steps of process may be carried out under purging with nitrogen. The pharmaceutical composition produced by the above steps may be filled in any containers, for instance, glass ampules, plastic ampules or prefilled syringes, after filter sterilization. Grass ampules or grass prefilled syringes are preferred, and preferably filled with nitrogen.

The pharmaceutical composition of the present invention may also be made into the final drug by combining with an appropriate pharmaceutical carrier or diluent and may be made into pharmaceutical preparations by any known common methods. For example, with regard to an injection preparation, it may be made into a solution or a suspension of an aqueous solvent or a non-aqueous solvent, such as distilled water for injection, physiological saline solution, Ringer's solution, vegetable oil, synthetic fatty acid glyceride, higher fatty acid ester and propylene glycol, because of osmotic pressure, water is preferred. In formulating the preparation, it may be made into a combination drug with other pharmaceutically active ingredient.

### Example 1

### Influence by Addition of Sodium Sulfite (1)

Monoammonium glycyrrhizinate, cysteine hydrochloride and aminoacetic acid were dissolved in water where the oxygen dissolved therein was small so as to make their compounding ratio 16 mg/mL (as glycyrrhizin), 8 mg/mL and 160 mg/mL, respectively. The solution was adjusted to pH from 7.2 to 7.5 with sodium hydroxide. After sodium sulfite was added thereto as a stabilizer so as to make 0, 2.4 or 4.0 mg/mL, the dissolved oxygen was removed using nitrogen. The solution was filtered, sterilized and filled in ampoules together with nitrogen. The ampoules were stored at 25° C. for four years and the state of precipitation of glycyrrhizin was observed. Separately, the ampoules were stored at 40° C. for four months or at 60° C. for 14 days and cysteine contained therein was quantified by an HPLC. In this manner, stability due to the difference in the adding amount of sodium sulfite in the pharmaceutical composition of the present invention was measured. An example of results is shown in Table 1.

TABLE 1

| | | | Adding amount of | | |
|---|---|---|---|---|---|
| | | | sodium sulfite | | |
| | | | (mg/mL) | | |
| | | | 0 | 2.4 | 4.0 |
| pH at manufacturing | | | 7.22 | 7.49 | 7.29 |
| Presence or | After 4 years | | − | + | + |
| absence of | at 25° C. | | | | |
| precipitation of | | | | | |
| glycyrrhizin | | | | | |
| Amount of cysteine | Before sterilization | | 97.3 | 101.6 | 98.9 |
| hydrochloride (%) | After sterilization | | 94.4 | 95.2 | 91.1 |
| | 60° C. | After 3 days | 89.7 | 87.8 | 81.4 |
| | | After 7 days | 81.2 | 71.3 | 64.2 |
| | | After 14 days | 77.8 | 66.5 | 53.9 |
| | 40° C. | After 2 months | 89.4 | 86.0 | 70.3 |
| | | After 4 months | 83.6 | 77.7 | 68.5 |

### Example 2

### Influence by Addition of Sodium Sulfite (2)

In the same manner as in the above Example 1, monoammonium glycyrrhizinate, cysteine hydrochloride and aminoacetic acid were dissolved in water where the oxygen dissolved therein was small so as to make their compounding ratio 16 mg/mL (as glycyrrhizin), 8 mg/mL and 160 mg/mL, respectively. The solution was adjusted to pH 7.2 to 7.5 with sodium hydroxide. Then there were prepared a product to which 6.4 mg/mL of sodium sulfite as a stabilizer was added and that to which no sodium sulfite was added. Each of them was also diluted into preparations so as to make concentrations of cysteine hydrochloride 6 mg/mL and 4 mg/mL. Oxygen dissolved therein was removed from them and the solution was filtered, sterilized and filled in ampoules with nitrogen. The ampoules were stored at 60° C. for 14 days and, at the stages of initiation and after 4 days, 7 days and 14 days, cysteine contained therein was quantified by an HPLC. In this manner, stability due to the difference in the adding amount of sodium sulfite in the pharmaceutical composition of the present invention was measured. An example of the results is shown in Table 2.

TABLE 2

| | | Concentration of cysteine | | |
|---|---|---|---|---|
| | | hydrochloride (mg/mL) | | |
| | | 8 | 6 | 4 |
| Non-addition | Initial | 100.0 | 100.0 | 100.0 |

| | | | | |
|---|---|---|---|---|
| 4 | days | 93.5 | 97.5 | 94.3 |
| 7 | days | 88.6 | 94.5 | 89.5 |
| 14 | days | 82.8 | 82.4 | 88.7 |

| | | | | |
|---|---|---|---|---|
| Addition of | Initial | 100.0 | 100.0 | 100.0 |

| | | | | | |
|---|---|---|---|---|---|
| sodium | 4 | days | 73.6 | 84.4 | 88.8 |
| sulfite | 7 | days | 53.1 | 70.4 | 77.1 |
| | 14 | days | 40.4 | 52.2 | 59.8 |

### Example 3

### Influence of pH on Gelling of Glycyrrhizic Acid

1) Production of a Glycyrrhizin-Containing Composition of pH 6

Predetermined amounts of sodium hydroxide, monoammonium glycyrrhizinate, cysteine hydrochloride monohydrate and aminoacetic acid as shown in Table 3 were successively dissolved in water (No hydrosulfite was added.). After that, pH was adjusted to 6.0 using 8 mol/L of sodium hydroxide solution or 6 mol/L of hydrochloric acid, and then adjusted to give a predetermined final volume. All of the above steps were conducted under purging with nitrogen. Two hundred mL thereof was sampled, sterilized by filtration using a 0.22-μm filter and filled in glass ampoules after purging with nitrogen until oxygen concentration reached 2 ppm or less using a monitor for dissolved oxygen.

TABLE 3

| Constituent | 1-fold | | 4-fold | | 5-fold | | 6-fold | | 8-fold | |
|---|---|---|---|---|---|---|---|---|---|---|
| Sodium hydroxide | 0.6 | g | 3.0 | g | 3.7 | g | 4.6 | g | 6.2 | g |
| Monoammonium | 4.1 | g | 16.4 | g | 20.5 | g | 24.6 | g | 32.8 | g |
| glycyrrhizinate | | | | | | | | | | |
| L-cysteine | 1.7 | g | 6.8 | g | 8.5 | g | 10.2 | g | 13.6 | g |
| hydrochloride | | | | | | | | | | |
| monohydrate | | | | | | | | | | |
| aminoacetic | 30.4 | g | 120.2 | g | 150.1 | g | 180.6 | g | 240.8 | g |
| acid | | | | | | | | | | |
| Final volume | 1.5 | L | 1.5 | L | 1.5 | L | 1.5 | L | 1.5 | L |

2) Production of Glycyrrhizin-Containing Compositions of Other pH Values

The residual liquid after the above 1) was successively adjusted to pH 5.5, 5.0 and 4.5 using 6 mol/L of hydrochloric acid and each 200 mL thereof was sampled. It was sterilized by filtration using a 0.22-μm filter and filled in glass ampoules after purging with nitrogen until oxygen concentration reached 2 ppm or less using a monitor for dissolved oxygen.

3) Storage, Property and Impurity Test of the Specimen

Each composition was stored at room temperature or in a cool place and its property and impurity were tested.

4) Results

A composition which was in the same ingredient concentration (1-fold) as the conventional product (Stronger Neo-Minophagen C) was gelled upon its production at pH 4.5. Compositions of 4-fold, 5-fold and 6-fold concentrations were gelled when stored in a cool place at pH 5.0. In the case of composition of 8-fold concentration, aminoacetic acid is crystallized out when stored in a cool place at pH 6.0. As shown in a footnote of Table 4, foaming upon purging with nitrogen at pH 5.5 was strong when a concentration was 4-fold or more whereupon the preparation thereof was difficult. When pH was 6.0, no strong foaming was generated in any of the concentrations. Table 4 shows the property of each composition being observed after storing at room temperature or in a cool place.

TABLE 4

| | Concentration of Composition | | | | |
|---|---|---|---|---|---|
| pH | 1-fold | 4-fold | 5-fold | 6-fold | 8-fold |
| 6.0 | not gelled | not gelled | not gelled | not gelled | Aminoacetic acid |
| | | | | | crystallization |
| | | | | | when stored |
| | | | | | in cool place |
| 5.5 | not gelled | not gelled* | not gelled* | not gelled* | Aminoacetic acid |
| | | | | | crystallization |
| | | | | | when stored |
| | | | | | in cool place |
| 5.0 | not gelled | gelled when | gelled when | gelled when | gelled when |
| | | stored in | stored in | stored in | stored in |
| | | cool place | cool place | cool place | cool place |
| 4.5 | gelled upon | gelled upon | gelled upon | gelled upon | gelled upon |
| | production | production | production | production | production |

*Foaming upon purging with nitrogen was so strong that the production was difficult

In view of the above result, stability test for cysteine under the hereinafter pH was investigated within a range of pH 6.0 to 7.5.

### Example 4

### Influence of pH on the Stability of Cysteine

### 1) Production of the Composition Including Glycyrrhizin

A composition which was in the same ingredient concentration (1-fold) as the conventional product (Stronger Neo-Minophagen C) and compositions in concentrations of 4-fold, 5-fold, 6-fold and 8-fold thereof were manufactured according to the following steps.

4 L of water for injection, and sodium hydroxide and monoammonium glycyrrhizinate in the amounts as shown in Table 5, were poured into a container for manufacture, and the components were dissolved in the water. Then, L-cysteine hydrochloride monohydrate, in an amount as shown in Table 5, was poured into the container and dissolved. After that, aminoacetic acid, in an amount shown in Table 5, was poured into the container and dissolved. The pH of the solution was adjusted to a pH of 7.5 using sodium hydroxide or 6 mol/L hydrochloric acid, and then the total volume of the solution was adjusted to 6 L.

After completing each of the above-described steps, the concentration of the dissolved oxygen (DO) was measured using a DO meter (B505 manufactured by Iijima Denshi Kogyo), and the results are shown in Table 5. All of the above steps were carried out under purging with nitrogen. However, as the evidence demonstrates, oxygen was present in the solution.

After adjusting each container to 6 L, about 1.2 L of the resulting solution was filtered using a filter (PVDF; diameter was 70 mm, pore size of 0.22 μm) and placed in an air-tight container. The headspace was substituted with nitrogen followed by storing the solution until the solution was filled in ampoules. When ready to be filled in ampoules, the resulting solution was transferred to a storage container for an automatic ampoule filling machine, purging of air was conducted with nitrogen and, after confirming that the dissolved oxygen concentration was 2 mg/L or less, 3 mL of each solution was filled into individual 3-mL glass ampoules using the automatic ampoule filling machine. Air in the ampoule was substituted with nitrogen and the ampoule was sealed by melting.

The remaining solution (about 4.8 L) in the container for manufacture was adjusted to a predetermined pH using 6 mol/L hydrochloric acid, in an amount as shown in Table 6. About 1.2 L thereof was similarly filled in glass ampoules and the above steps were repeated to manufacture each of the preparations of pH 7.0, 6.5 and 6.0. Dissolved oxygen concentration in each preparation upon filling in an ampoule is shown in Table 6.

TABLE 5

| | Concentration | | | | | | | | | | |
|---|---|---|---|---|---|---|---|---|---|---|---|
| Step | Factor | 1-fold | | 4-fold | | 5-fold | | 6-fold | | 8-fold | |
| Dissolution of | Amount of | 4 | L | 4 | L | 4 | L | 4 | L | 4 | L |
| sodium | water for | | | | | | | | | | |
| hydroxide | injection | | | | | | | | | | |
| | Poured amount | 4.06 | g | 15.02 | g | 17.95 | g | 21.32 | g | 28.44 | g |
| | Dissolved | 3.72 | mg/L | 3.05 | mg/L | 3.14 | mg/L | 3.30 | mg/L | 3.10 | mg/L |
| | oxygen | | | | | | | | | | |
| | concentration | | | | | | | | | | |
| Dissolution of | Poured amount | 17.47 | g | 69.86 | g | 87.36 | g | 104.85 | g | 139.81 | g |
| monoammonium | Dissolved | 1.81 | mg/L | 1.88 | mg/L | 1.69 | mg/L | 1.37 | mg/L | 1.95 | mg/L |
| glycyrrhizinate | oxygen | | | | | | | | | | |
| | concentration | | | | | | | | | | |
| Dissolution of | Poured amount | 6.70 | g | 26.82 | g | 33.46 | g | 40.33 | g | 53.53 | g |
| L-cysteine | Dissolved | 2.16 | mg/L | 1.74 | mg/L | 1.59 | mg/L | 1.20 | mg/L | 1.31 | mg/L |
| hydrochloride | oxygen | | | | | | | | | | |
| monohydrate | concentration | | | | | | | | | | |
| Dissolution of | Poured amount | 121.11 | g | 484.75 | g | 605.31 | g | 726.44 | g | 968.52 | g |
| aminoacetic | Dissolved | 1.98 | mg/L | 1.95 | mg/L | 1.76 | mg/L | 1.62 | mg/L | 1.97 | mg/L |
| acid | oxygen | | | | | | | | | | |
| | concentration | | | | | | | | | | |
| Adjustment of pH | Sodium | — | | 0.5 | g | 0.6 | g | 0.8 | g | 1.2 | g |
| and | hydroxide | | | | | | | | | | |
| adjustment of | 6 mol/L | 0.25 | mL | — | | — | | — | | — | |
| liquid amount | Hydrochloric | | | | | | | | | | |
| | acid | | | | | | | | | | |
| | Adjusted | 6 | L | 6 | L | 6 | L | 6 | L | 6 | L |
| | liquid amount | | | | | | | | | | |
| | Dissolved | 1.56 | mg/L | 1.39 | mg/L | 1.04 | mg/L | 0.99 | mg/L | 0.91 | mg/L |
| | oxygen | | | | | | | | | | |
| | concentration | | | | | | | | | | |

TABLE 6

| | Concentration | | | | | |
|---|---|---|---|---|---|---|
| Step | Factor | 1-fold | 4-fold | 5-fold | 6-fold | 8-fold |
| Filling | pH upon | pH | pH | pH | pH | pH |
| of pH | preparation | 7.50 | 7.50 | 7.50 | 7.51 | 7.50 |
| 7.5 | Dissolved | 0.82 mg/L | 0.68 mg/L | 0.99 mg/L | 0.88 mg/L | 1.53 mg/L |
| | oxygen | | | | | |
| | concentration | | | | | |
| Filling | 6 mol/L | 1.75 mL | 6.7 mL | 6.7 mL | 8.0 mL | 11.0 mL |
| of pH | Hydrochloric | | | | | |
| 7.0 | acid | | | | | |
| | pH upon | pH | pH | pH | pH | pH |
| | preparation | 7.00 | 7.00 | 6.99 | 7.00 | 7.01 |
| | Dissolved | 0.83 mg/L | 0.74 mg/L | 0.84 mg/L | 0.94 mg/L | 0.50 mg/L |
| | oxygen | | | | | |
| | concentration | | | | | |
| Filling | 6 mol/L | 0.5 mL | 2.0 mL | 2.2 mL | 2.8 mL | 4.0 mL |
| of pH | Hydrochloric | | | | | |
| 6.5 | acid | | | | | |
| | pH upon | pH | pH | pH | pH | pH |
| | preparation | 6.49 | 6.50 | 6.50 | 6.49 | 6.50 |
| | Dissolved | 0.75 mg/L | 0.46 mg/L | 0.72 mg/L | 0.94 mg/L | 0.50 mg/L |
| | oxygen | | | | | |
| | concentration | | | | | |
| Filling | 6 mol/L | 0.25 mL | 1.0 mL | 1.6 mL | 2.0 mL | 3.3 mL |
| of pH | Hydrochloric | | | | | |
| 6.0 | acid | | | | | |
| | pH upon | pH | pH | pH | pH | pH |
| | preparation | 6.00 | 6.00 | 6.00 | 6.00 | 6.00 |
| | Dissolved | 0.92 mg/mL | 0.51 mg/mL | 0.57 mg/mL | 0.86 mg/mL | 0.78 mg/mL |
| | oxygen | | | | | |
| | concentration | | | | | |

### 2) Stability Test of L-Cysteine Hydrochloride

The manufactured preparations were stored in a chamber of constant temperature and humidity at 40° C. and 75% RH and, after six months, the amount of L-cysteine in each preparation was quantified by means of high-performance liquid chromatography (HPLC) to determine the residual rate of L-cysteine.

### 3) Results

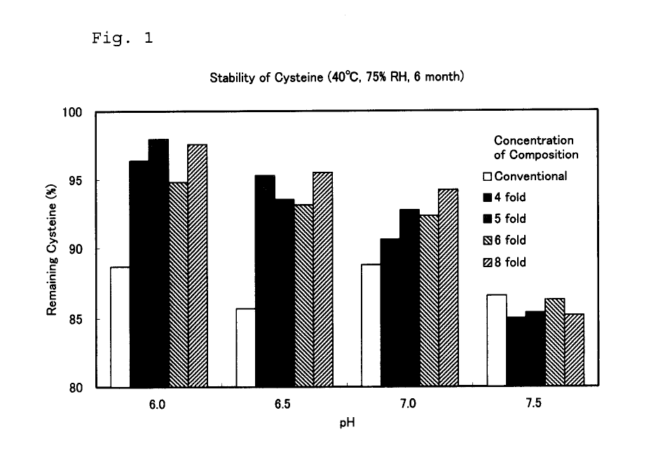

The results of the stability test of L-cysteine hydrochloride (at 40° C. and 75% RH for six months) are shown in FIG. 1. In the preparation of the conventional product, the residual rate of L-cysteine was lower than 90% at any pH. On the contrary, in the preparations at concentrations of 4- to 8-fold, the residual rates of L-cysteine at pH 6.0 to 7.0 were significantly and unexpectedly higher than that of the preparation in the concentration of the conventional product. However, at pH 7.5, in the preparations at concentrations of 4- to 8-fold, the residual rates of L-cysteine was lower than 90% and there was no difference from the preparation in the concentration of the conventional product.

The results depicted in FIG. 1 demonstrate that when the concentration of the preparation containing no sulfite is made as high as 4- to 8-fold, stability of L-cysteine hydrochloride at a pH of 6.0 to 7.0 is significantly and unexpectedly increases compared to a preparation having a conventional concentration of components. In addition, the results demonstrate that even though purging of the air was done with nitrogen, oxygen remained in each of the solutions. Therefore, the unexpected increase in the stability of cysteine is not due to the absence of oxygen, but is due to the high concentration of the components of the solution.

### 4) Osmotic Pressure of Each Pharmaceutical Composition

The results of osmotic pressure of the pharmaceutical preparations of pH 6 or 7.5 produced at 1) measured by an osmometer were demonstrated in Table 7

TABLE 7

| | | Ratio of osmotic |
|---|---|---|
| | | pressure compare |
| pH | Concentration | to saline |
| pH 6.0 | 1-fold | 1.0 |
| | 4-fold | 4.1 |
| | 5-fold | 5.0 |
| | 6-fold | 5.9 |
| | 8-fold | 8.0 |
| pH 7.5 | 1-fold | 1.0 |
| | 4-fold | 4.1 |
| | 5-fold | 4.9 |
| | 6-fold | 5.9 |
| | 8-fold | 7.9 |

| | |
|---|---|
| saline | 1.0 |

### INDUSTRIAL APPLICABILITY

As apparent from the result shown in the aforementioned Tables 1 and 2, the higher the sodium sulfite concentration is, the more the reduction in the amount of cysteine with lapse of time is. In the cases where sodium sulfite was added, precipitate of glycyrrhizin was produced while, in the cases where no sodium sulfite was added, not only precipitate of glycyrrhizin which was added in a high concentration was not produced but also reduction in the amount of cysteine was low whereby stability was improved. As shown in Table 4, aminoacetic acid may be crystallized upon storing in a cool place in a composition of 8-fold concentration whereby it is shown that the concentrations of 4-fold to 6-fold were preferred. When pH was lower than 6.0, gelling of glycyrrhizin or, due to the viscosity, foaming upon purging with nitrogen was strong and that was not preferred. Further, as shown in FIG. 1, residual rates of cysteine at pH 6.0 to 7.0 in the compositions of 4- to 8-fold concentrations were significantly higher than that in the composition of the conventional product concentration but, when pH was 7.5, residual rate of cysteine lowered and no difference was noted between the above and that in the composition of a concentration being the same as that of the conventional product. As such, in the preparation of the present invention where glycyrrhizin was concentrated, amounts of effective ingredients are in higher concentrations than in the conventional preparations and both stability and safety are also excellent whereby its utility as a pharmaceutical composition is very high.

1. An injectable pharmaceutical composition consisting essentially of 8 to 12 mg/mL of glycyrrhizin, 3 to 5 mg/mL of cysteine and 80 to 120 mg/mL of aminoacetic acid,
wherein substantially no sulfite is contained in the pharmaceutical composition,
wherein the glycyrrhizin, cysteine and aminoacetic acid are dissolved in water, and
wherein a pH of the pharmaceutical composition is 6 to 7.

2. The injectable pharmaceutical composition according to claim 1, wherein the glycyrrhizin is monoammonium glycyrrhizinate.

3. The injectable pharmaceutical composition according to claim 1, wherein the cysteine is cysteine hydrochloride.

4. The injectable pharmaceutical composition according to claim 1, wherein the concentration of cysteine in the pharmaceutical composition after the composition is stored at 40° C. and 75% relative humidity for 6 months is more than 90% of an initial concentration of cysteine in the pharmaceutical composition.

5. The injectable pharmaceutical composition according to claim 1, wherein the pharmaceutical composition consists essentially of 10 mg/mL of glycyrrhizin, 4 mg/mL of cysteine and 100 mg/mL of aminoacetic acid.

6. The injectable pharmaceutical composition according to claim 1, wherein the pharmaceutical composition is intravenously injected.
